(19) United States
(12) Patent Application Publication
(10) Pub. No.: US 2023/0158067 A1
(43) Pub. Date: May 25, 2023

(54) SILVER NANOPARTICLES FOR USE IN INHIBITING AND TREATING CORONAVIRUS INFECTION

(71) Applicant: Ankit Agarwal, Middleton, WI (US)

(72) Inventor: Ankit Agarwal, Middleton, WI (US)

(21) Appl. No.: 17/917,122

(22) PCT Filed: Apr. 6, 2021
(86) PCT No.: PCT/US2021/025942

§ 371 Date: Oct. 5, 2022

Related U.S. Application Data
(60) Provisional application No. 63/005,727, filed on Apr. 6, 2020.

(51) Int. Cl. A61K 33/38; A61K 9/14; A61K 9/00; A61P 31/14

(52) U.S. Cl. CPC A61K 33/38; A61K 9/14; A61K 9/0078; A61P 31/14

(57) ABSTRACT

The present invention relates to the use of compositions comprising sialic acid to inhibit or treat coronavirus infections, and in particular those caused by SARS-CoV-2 (Severe Acute Respiratory Syndrome Coronavirus 2).

### CROSS REFERENCE TO RELATED APPLICATION

This application claims the benefit of U.S. Prov. Appl. 63/005,727, filed Apr. 6, 2020 which is incorporated by reference herein in its entirety.

### FIELD OF THE INVENTION

The present invention relates to the use of silver nanoparticles to inhibit or treat coronavirus infections, and in particular those caused by SARS-CoV-2 (Severe Acute

Respiratory Syndrome Coronavirus 2).

### BACKGROUND OF THE INVENTION

Coronaviruses are a family of viruses that can cause illnesses such as the common cold, severe acute respiratory syndrome (SARS) and Middle East respiratory syndrome (MERS). In 2019, a new coronavirus was identified as the cause of a disease outbreak that originated in China. The virus is now known as the severe acute respiratory syndrome coronavirus 2 (SARS-CoV-2). The disease it causes is called coronavirus disease 2019 (COVID-19). Cases of COVID-19 have been reported around the world and WHO declared a global pandemic in March 2020.

Signs and symptoms of COVID-19 may appear two to 14 days after exposure and can include: fever; cough; and shortness of breath or difficulty breathing. Other symptoms can include: tiredness; aches; runny nose; and sore throat. The severity of COVID-19 symptoms can range from very mild to severe. Some people have no symptoms. People who are older or have existing chronic medical conditions, such as heart or lung disease or diabetes, may be at higher risk of serious illness.

What is needed in the art are safe compositions for inhibiting or treating infection by SARS-CoV-2.

### SUMMARY OF THE INVENTION

The present invention relates to the use of silver nanoparticles to inhibit or treat respiratory virus infections, and in particular those caused by SARS-CoV-2 (Severe Acute Respiratory Syndrome Coronavirus 2).

Accordingly, in some preferred embodiments, the present invention provides methods for treating or inhibiting infection by a respiratory virus, in a human or animal subject, the method comprising administering silver nanoparticles in an effective concentration to the subject under conditions such that infection by the respiratory virus is inhibited or treated. In some preferred embodiments, the present invention provides methods for prophylaxis of infection by a respiratory virus, in a human or animal subject, the method comprising: administering silver nanoparticles in an effective concentration to the subject under conditions such that infection by the respiratory virus is inhibited. In some preferred embodiments, the present invention provides silver nanoparticles for use in treating or inhibiting infection by a respiratory virus in a human or animal subject. In some preferred embodiments, the present invention provides silver nanoparticles for use in prophylaxis of respiratory virus infection in a human or animal subject. In some preferred embodiments, the respiratory virus is selected from the group consisting of influenza virus, respiratory syncytial virus, a parainfluenza virus, a herpes virus, metapneumovirus, rhinovirus, a coronavirus, an adenoviruses, and a bocavirus. In some preferred embodiments, the coronavirus is SARS CoV 2 (Severe Acute Respiratory Syndrome Coronavirus 2). In some preferred embodiments, the subject is at risk for infection by SARS-CoV-2. In some preferred embodiments, the subject has COVID-19. Other viruses that may be treated or inhibited include Human immunodeficiency virus (HIV-1).

In some preferred embodiments, the silver nanoparticles are from 1 to 100 nm in size. In some preferred embodiments, the silver nanoparticles are provided in a formulation and the average size of the nanoparticles in the formulation are from 1 to 50 nm in size. In some preferred embodiments, the average size of the nanoparticles in the formulation is from 1 to 10 nm in size. In some preferred embodiments, the silver nanoparticles are formulated in a suspension. In some preferred embodiments, the suspension is an inhalation suspension. In some preferred embodiments, the concentration of silver nanoparticles in the suspension is from 0.01 to 200 μg/ml. In some preferred embodiments, the concentration of silver nanoparticles in the suspension is from 0.1 to 100 μg/ml. In some preferred embodiments, the concentration of silver nanoparticles in the suspension is from 0.01 to 5 μg/ml. 32. In some preferred embodiments, the silver nanoparticles are stabilized with starch. In some preferred embodiments, the silver nanoparticles are prepared by reduction of a silver nitrate salt with tannic acid.

In some preferred embodiments, the silver nanoparticles are formulated for intranasal administration. In some preferred embodiments, the silver nanoparticles are formulated with one or more physiologically acceptable carriers. In some preferred embodiments, the silver nanoparticles are stabilized to prevent agglomeration. In some preferred embodiments, the silver nanoparticles are formulated in an aqueous suspension for use in a nebulizer or misting apparatus. In some preferred embodiments, the silver nanoparticles are delivered to the lung of a subject via inhalation. In some preferred embodiments, the inhalation is via a continuous nebulizer. In some preferred embodiments, the silver nanoparticles are formulated in an aqueous suspension for use as an aerosol. In some preferred embodiments, the silver nanoparticles are formulated as a nasal spray. In some preferred embodiments, the silver nanoparticles are formulated with thixotropic agent. In some preferred embodiments, the silver nanoparticles are delivered to the lung of a subject via intranasal administration. In some preferred embodiments, an aqueous suspension of the silver nanoparticles has a surface plasmon peak between 400 and 420 nm. In some preferred embodiments, the dosage for inhalation is from 0.5 ml to 10 ml of a aqueous suspension of from 10 to 200 μg/ml silver nanoparticles. In some preferred embodiments, the dosage is administered from 1 to 5 times daily. In some preferred embodiments, the dosage is administered 3 times daily. In some preferred embodiments, the dosage is administered for from 5 to 20 days.

In some preferred embodiments, the present invention provides methods for treating or inhibiting infection by SARS-CoV-2 (Severe Acute Respiratory Syndrome Coronavirus 2), in a human or animal subject, the methods comprising: administering silver nanoparticles in an effective concentration to the subject under conditions such that infection by SARS-CoV-2 is inhibited or treated.

In some preferred embodiments, the present invention provides methods for prophylaxis of infection by SARS-CoV-2 (Severe Acute Respiratory Syndrome Coronavirus 2), in a human or animal subject, the methods comprising: administering silver nanoparticles in an effective concentration to the subject under conditions such that infection by SARS-CoV-2 is inhibited.

In some preferred embodiments, the present invention provides silver nanoparticles for use in treating or inhibiting infection by SARS-CoV-2 (Severe Acute Respiratory Syndrome Coronavirus 2) in a human or animal subject.

In some preferred embodiments, the present invention provides silver nanoparticles for use in prophylaxis of SARS-CoV-2 (Severe Acute Respiratory Syndrome Coronavirus 2) infection in a human or animal subject.

In some preferred embodiments, the silver nanoparticles are from 2 to 100 nm in size. In some preferred embodiments, the silver nanoparticles are provided in a formulation and the average size of the nanoparticles in the formulation are from 2 to 50 nm in size. In some preferred embodiments, the average size of the nanoparticles in the formulation is from 5 to 15 nm in size. In some preferred embodiments, the average size of the nanoparticles in the formulation is from 1 to 10 nm in size. In some preferred embodiments, the average size of the nanoparticles in the formulation is from 1 to 5 nm in size. In some preferred embodiments, the average size of the nanoparticles in the formulation is from 2 to 10 nm in size. In some preferred embodiments, the average size of the nanoparticles in the formulation is from 2 to 5 nm in size. In some preferred embodiments, the silver nanoparticles are formulated in a suspension. In some preferred embodiments, the concentration of silver nanoparticles in the suspension is from 1 to 200 μg/ml. In some preferred embodiments, the concentration of silver nanoparticles in the suspension is from 1 to 100 μg/ml. In some preferred embodiments, the concentration of silver nanoparticles in the suspension is from 5 to 50 μg/ml. In some preferred embodiments, the concentration of silver nanoparticles in the suspension is from 0.01 to 200 μg/ml. In some preferred embodiments, the concentration of silver nanoparticles in the suspension is from 0.01 to 100 μg/ml. In some preferred embodiments, the concentration of silver nanoparticles in the suspension is from 0.01 to 10 μg/ml. In some preferred embodiments, the concentration of silver nanoparticles in the suspension is from 0.01 to 5 μg/ml. In some preferred embodiments, the concentration of silver nanoparticles in the suspension is from 0.1 to 200 μg/ml. In some preferred embodiments, the concentration of silver nanoparticles in the suspension is from 0.1 to 100 μg/ml. In some preferred embodiments, the concentration of silver nanoparticles in the suspension is from 0.1 to 10 μg/ml. In some preferred embodiments, the concentration of silver nanoparticles in the suspension is from 0.1 to 5 μg/ml.

In some preferred embodiments, the administration of the silver nanoparticle composition is sufficient to relieve or ameliorate one or more symptoms of COVID-19. The symptoms that are relieved or ameliorated include one or more of fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory. In some preferred embodiments, administration of the silver nanoparticle composition improves or alleviates one or more of the following symptoms in a long COVID patient: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory.

In some preferred embodiments, the silver nanoparticles are formulated for intranasal administration. In some preferred embodiments, the silver nanoparticles are formulated with one or more physiologically acceptable carriers. In some preferred embodiments, the silver nanoparticles are stabilized to prevent agglomeration. In some preferred embodiments, the silver nanoparticles are formulated as a nasal spray. In some preferred embodiments, the silver nanoparticles are formulated with thixotropic agent. In some preferred embodiments, the silver nanoparticles are formulated in a suspension for use in a nebulizer or misting apparatus. In some preferred embodiments, the silver nanoparticles are formulated in a suspension for use as an aerosol. In some preferred embodiments, the silver nanoparticles are formulated in a suspension for use in a nasal spray device. In some preferred embodiments, the silver nanoparticles are delivered to the lung of a subject via intranasal administration. In some preferred embodiments, mist of an aqueous suspension of the silver nanoparticles is inhaled into the lungs of a subject via a nebulizer. In some preferred embodiments, the subject is at risk for infection by SARS-CoV-2. In some preferred embodiments, the subject has COVID-19. In some preferred embodiments, the silver nanoparticles are stabilized with starch. In some preferred embodiments, the silver nanoparticles are prepared by reduction of a silver nitrate salt with tannic acid.

### BRIEF DESCRIPTION OF THE DRAWINGS

FIG. 1 provides data related to suspensions of silver nanoparticle suspensions of the present invention. FIG. 1 (A) Clear dark brown suspension of stabilized AgNP; FIG. 1 (B) UV-Vis spectra of AgNP suspension; FIG. 1 (C) TEM imaging showing 2-10 nm dia AgNP; scale bar 10 nm; FIG. 1 (D) size distribution of AgNP in TEM imaging.

### DEFINITIONS

As used herein, the term “SARS-CoV-2 (severe acute respiratory syndrome coronavirus 2)” includes any strain of coronavirus identified as being SARS-CoV-2 including mutants of SARS-CoV-2 reference genomic sequences.

As used herein, the term “silver nanoparticle(s)” refers are nanoparticles of silver which are in the range of between 1 and 100 nm in at least one dimension.

As used herein, the term “inhibits” when used in reference to infection by SARS-CoV-2 refers to a reduction in infection in subjects exposed to SARS-CoV-2.

A “patient,” “subject,” or “individual” are used interchangeably and refer to either a human or a non-human animal. These terms include mammals, such as humans, primates, livestock animals (including bovines, swine, etc.), companion animals (e.g., canines, felines, etc.) and rodents (e.g., mice and rats).

“Administering” or “administration of” a substance, a compound or an agent to a subject can be carried out using one of a variety of methods known to those skilled in the art. For example, silver nanoparticles can be administered intranasally and a mist of an aqueous suspension of silver nanoparticles can be inhaled into the lungs using a hand-held continuous nebulizer. A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

A “therapeutically effective amount” or a “therapeutically effective dose” of a drug or agent, such as silver nanoparticles, is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of symptoms of the condition being treated, such as COVID-19. The skilled worker can readily determine the effective amount for a given situation by routine experimentation.

A “prophylactically effective amount” or a “prophylactically effective dose” of a drug or agent, such as silver nanoparticles, is an amount of a drug or an agent that, when administered to a subject will have the intended prophylactic effect. The full prophylactic effect does not necessarily occur by administration of one dose, and may occur only after administration of a series of doses. Thus, a prophylactically effective amount may be administered in one or more administrations. The precise effective amount needed for a subject will depend upon, for example, the subject's size, health and age, the nature and extent of symptoms of the condition being treated, such as SARS-CoV-2 infection. The skilled worker can readily determine the effective amount for a given situation by routine experimentation.

“Treating” a condition or patient refers to taking steps to obtain beneficial or desired results, including clinical results. Beneficial or desired clinical results include, but are not limited to, alleviation, amelioration, or slowing the progression, of one or more symptoms associated with COVID-19. In certain embodiments, treatment may be prophylactic, such as for the prevention of infection by SARS-CoV-2.

### DETAILED DESCRIPTION OF THE INVENTION

The present invention relates to the use of silver nanoparticles to inhibit or treat respiratory virus infections, and in particular those caused by SARS-CoV-2 (Severe Acute Respiratory Syndrome Coronavirus 2).

To date, viruses, causing respiratory infectious diseases, have become a global threat to the human health by affecting approximately 9% of the world's population annually and killing up to 500,000 people each year. See Clayville, L. R., Influenza update: a review of currently available vaccines. P T, 2011. 36(10): p. 659-84; Renukaradhya, G. J., B. Narasimhan, and S. K. Mallapragada, Respiratory nanoparticle-based vaccines and challenges associated with animal models and translation. J Control Release, 2015. 219: p. 622-631. These viruses include influenza virus (H1N1, H3N2, etc.), respiratory syncytial virus (RSV), severe acute respiratory syndrome (SARS), Middle East respiratory syndrome (MERS) and newly emerging strains such as severe acute respiratory syndrome coronavirus 2 (SARS-CoV2). See Morris, D., et al., Antiviral and Immunomodulatory Activity of Silver Nanoparticles in Experimental RSV Infection. Viruses, 2019. 11(8). Most of these viruses are highly contagious and responsible for severe morbidity and mortality. See Xiang, D., et al., Inhibition of A/Human/Hubei/3/2005 (H3N2) influenza virus infection by silver nanoparticles in vitro and in vivo. Int J Nanomedicine, 2013. 8: p. 4103-13; Alghrair, Z. K., D. G. Fernig, and B. Ebrahimi, Enhanced inhibition of influenza virus infection by peptide-noble-metal nanoparticle conjugates. Beilstein J Nanotechnol, 2019. 10: p. 1038-1047.

Upon entering the human body by airways, they start the infection by attaching to the mucosal layer, then infecting epithelium of upper respiratory tract and quickly spread to the lower respiratory tract by intracellular transmission. In general, the symptoms of the infection range from the low fever to severe bronchitis, or, pneumonia, which mostly lead to the death of the infected patients. Researchers around the world have been trying to create vaccines to overcome these pandemic diseases, but these vaccines take ample amount of time and optimizations before they start treating people. On the other side, many researchers have been working on creating antiviral drugs such as monoclonal antibodies (MABs), proteases to limit the transmission of the viruses to nearby cells. However, these antiviral drugs produce severe side-effects, which limits the use of these drugs to many people.

The present invention is not limited to the use of silver nanoparticles for the treatment or inhibition of infection any particular respiratory virus. Indeed, the present invention contemplated treatment of inhibition of infections a variety of respiratory viruses, including, but not limited to, influenza viruses (e.g., H1N1 and H3N2), respiratory syncytial virus (RSV), parainfluenza viruses, metapneumoviruses, herpes viruses (e.g., herpes simplex virus 2), rhinoviruses, coronaviruses (e.g., SARS CoV 1 and SARS CoV 2), adenoviruses, and bocaviruses. Other viruses that may be treated or inhibited include Human immunodeficiency virus (HIV-1).

Unlike other antiviral drugs which kills viruses through chemical interactions, silver nanoparticles affect these viruses through physical interactions. They first bind to sulfur-bearing residues on surface glycoproteins located on the outer viruses' capsid and thus prevent their attachment and entry into the host cell. Then, they block the cellular factors that are necessary for the proper assemble of the viral proteins. See Xiang, D. X., et al., Inhibitory effects of silver nanoparticles on H1N1 influenza A virus in vitro. J Virol Methods, 2011. 178(1-2): p. 137-42. Xiang et al. (2011 and 2013) investigated the beneficial effects of AgNPs in vitro on human Madin-Darby canine kidney (MDCK) cells and in vivo by delivering AgNPs intranasally to 8-10-week-old female BALB/c mice. They showed that AgNPs at concentration 50 μg/ml inhibited both H1N1 and H3N2 strains of influenza viruses and significantly reduced the apoptosis of MDCK cells. They also demonstrated that intranasally delivered AgNPs significantly increased the survival rate of mice, which were pre-infected with influenza virus. In another recently published study by Morris et al. (2019), they treated BALB/c mice infected with RSV by delivering AgNPs intranasally. It was the first in vivo experiment which demonstrated the antiviral properties of AgNPs against RSV. They showed that the significant reduction of pro-inflammatory cytokines (i.e., IL-la, IL-6, TNF-α) and pro-inflammatory chemokines (i.e., CCL2, CCL3, CCLS) in RSV-infected mice treated with AgNPs.

Researchers have shown some concerns of the AgNPs toxicity to human health. However, many studies have thoroughly explored the toxicity effects of AgNPs delivered intranasally in rodent models. They showed that AgNPs caused minor thickening of mucosal layer and cellular infiltration (primarily neutrophils), but caused no major alterations to physiological lung function, even following 28 days of continuous exposure. These studies have identified the broad-spectrum antiviral properties of silver nanoparticles (AgNPs) against respiratory pathogens, such as adenovirus, parainfluenza, and influenza. The primary objective of this invention is to develop a suspension of silver nanoparticles (AgNPs) and deliver them intranasally to the people infected with respiratory viruses.

The present invention provides a stable antiviral composition (preferably a suspension) that delivers silver nanoparticles (AgNPs) intranasally to inhibit the broad spectrum of respiratory viruses including SARS-CoV-2 and prevent the progression of viral transmission in people infected with viruses. It is contemplated that the silver nanoparticles inhibit viruses in the upper and/or lower respiratory tracts and increase the chances of survival of infected people. A scientific premise is that such inhaled silver nanoparticles would attach to respiratory viruses lodged in the upper and lower respiratory tracts, disrupt the viral morphological structure, block spike proteins from binding to receptors of host cells, and inhibit viral infection and replication.

There are many methods for synthesizing colloidal suspensions of stabilized silver nanoparticles using physical, chemical and biological methods, reviewed in detail elsewhere along with their properties and multifunctional biomedical applications. See Zhang X-F, Liu Z-G, Shen W, Gurunathan S. Silver Nanoparticles: Synthesis, Characterization, Properties, Applications, and Therapeutic Approaches. International Journal of Molecular Sciences. 2016; 17(9), incorporated herein by reference in its entirety.

Silver nanoparticles may be synthesized by physical and chemical processes. Silver nanomaterials can be obtained by both the so-called ‘top-down’ and ‘bottom-up’ methods. The top-down method involves the mechanical grinding of bulk metals and subsequent stabilization of the resulting nanosized metal particles by the addition of colloidal protecting agents. The bottom-up methods, on the other hand, include reduction of metals, electrochemical methods, and sonodecomposition.

In some embodiments, silver nanoparticles are synthesized by a chemical method of reduction of the metal salt AgBF₄ by NaBH₄ in water. Nanoparticles ranging in size, for example, from 3 to 40 nm may be made by this method. Nanoparticle quality may be assessed transmission electron microscopy (TEM) and/or UV-visible (UV-vis) absorption spectroscopy. In some preferred embodiments, the silver nanoparticle compositions or formulations of the present invention are characterized by the percent of silver particles in the composition having at least one dimension of 100 nm or less. In some preferred embodiments, at least 80% of the silver particles in the formulation or composition have at least one dimension such as diameter of less than 100 nm. In more preferred embodiments, at least 90% of the silver particles in the formulation or composition have at least one dimension such as diameter of less than 100 nm. In some still more preferred embodiments, at least 95% of the silver particles in the formulation or composition have at least one dimension such as diameter of less than 100 nm.

In other embodiments, silver nanoparticles are produced by the electrochemical method which involves the electroreduction of AgNO₃ in aqueous solution in the presence of polyethylene glycol. The nanoparticles thus produced can characterized by TEM, X-ray diffraction, and UV-vis absorption spectroscopy. In other preferred embodiments, sonodecomposition is used to produce silver nanoparticles and involves the usage of ultrasonic waves to induce cavitation, a phenomenon whereby the passage of ultrasonic waves through an aqueous solution yields microscopic bubbles that expand and ultimately burst. The synthesis of silver nanoparticles involves sonochemical reduction of an aqueous silver nitrate solution in an atmosphere of argon-hydrogen. The silver nanoparticles can be characterized by TEM, X-ray diffraction, absorption spectroscopy, differential scanning calorimetry, and/or EPR spectroscopy. In other preferred embodiments, microwave synthesis of silver nanoparticles is utilized, which involves the reduction of silver nanoparticles using variable frequency microwave radiation. Other preferred methods for producing silver nanoparticles include thermal decomposition in organic solvents, chemical and photoreduction in reverse micelles, spark discharg, and cryochemical synthesis.

In some preferred embodiments, the present invention employs synthesis of silver nanoparticles through reduction of silver ions inside the nanoscopic starch templates. See Lomeli-Marroquin D, Medina Cruz D, Nieto-Arguello A, Vernet Crua A, Chen J, Torres-Castro A, et al. Starch-mediated synthesis of mono- and bimetallic silver/gold nanoparticles as antimicrobial and anticancer agents. International journal of nanomedicine. 2019; 14:2171-90; Mohan S, Oluwafemi O S, George S C, Jayachandran V P, Lewu F B, Songca S P, et al. Completely green synthesis of dextrose reduced silver nanoparticles, its antimicrobial and sensing properties. Carbohydrate Polymers. 2014; 106:469-74; Raveendran P, Fu J, Wallen S L. Completely “Green” Synthesis and Stabilization of Metal Nanoparticles. Journal of the American Chemical Society. 2003; 125(46):13940-1; Yakout S M, Mostafa A A. A novel green synthesis of silver nanoparticles using soluble starch and its antibacterial activity. International journal of clinical and experimental medicine. 2015; 8(3):3538-44; each of which is incorporated by reference herein its entirety. The hydroxyl groups of starch act as passivation contacts for the stabilization of the nanoparticles formed inside these templates.

Preparation of nanoparticles involves the reduction of metals ions in solutions or in high temperature gaseous environments. The high surface energy of the nanoparticles makes them extremely reactive. Most systems undergo aggregations without protection or passivation of their surfaces. See Freeman R G, Grabar K C, Allison K J, Bright R M, Davis J A, Guthrie A P, et al. Science. 1995; 267:1629; Ullman A. Chem Rev. 1996; 96:1533; Zhao M, Sun L, Crooks R M. J Am Chem Soc. 1998; 120:4877; Wang R, Yang J, Zheng Z, Carducci M D, Jiao J, Seraphin S. Angew Chem, Int Ed. 2001; 40:549; Zheng J, Stevenson M S, Hikida R S, Patten P G V. J Phys Chem B. 2002; 106:1252; each of which is incorporated by reference herein its entirety. Commonly used methods for surface passivation include protection by self-assembled monolayers, the most popular being thiol-functionalized organics, encapsulation in the H₂O pools of reverse microemulsions, and dispersion in polymeric matrices. See Petit C, Lixon P, Pileni M. J Phys Chem B. 1993; 97:12974; Suslick K S, Fang M, Hyeon T. J Am Chem Soc. 1996; 118:11960; each of which is incorporated herein by reference in its entirety. Moreover, majority of these methods use strong reducing agents such as hydrazine, sodium borohydride, and dimethyl formamide. These are highly reactive chemicals that pose biological risks.

In some preferred embodiments, the reducing sugar, β-D-glucose is employed as the reducing agent. See Raveendran et al., cited above. β-D-glucose is a mild, renewable, non-toxic reducing agent. To protect and passivate the nanoparticle surface, capping material employed is starch. The choice of capping material depends on the desired size and morphology of the nanoparticles in the targeted application. Linear as well as dendritic polymers have been successfully used for nanoparticle synthesis. See Suslick K S, Fang M, Hyeon T. J Am Chem Soc. 1996; 118:11960 and Zhao M, Sun L, Crooks R M. J Am Chem Soc. 1998; 120:4245, each of which is incorporated herein by reference in its entirety. Starch, in particular amylose, has an extensive number of hydroxyl groups that facilitate the complexation of silver ions to the molecular matrix. In turn, silver ions could also guide supramolecular organization among starch molecules.

A key advantage of using starch as the protecting agent is that it is completely soluble in water and, thus, does not require use of organic solvents—making it readily available for biomedical applications. Moreover, the binding between starch and metal nanoparticles is relatively weak compared to thiol-based protecting groups. This implies that the protection should be easily reversible at relatively high temperatures, enabling the separation of these particles. Additionally, place exchange reactions can be used to functionalize the nanoparticles. See Templeton A C, Chen S, Cross S M, Murray R W. Langmuir. 1999; 15:66, incorporated by reference herein in its entirety.

The present invention is not limited to any particular method of preparing silver nanoparticles. As an exemplary embodiment, a 0.1M solution of silver nitrate and a 0.17 wt % aqueous solution of soluble starch are prepared. A 100 uL aliquot of silver nitrate solution is added to 6 mL starch solution. After complete dissolution, a 150 uL aliquot of 0.1M aqueous solution of β-D-glucose is added with stirring. The mixture is heated to 40° C. and maintained at this temperature for 20 h. All solution components are preferably purged with argon before use and reduction proceeds in presence of argon to eliminate oxygen. The solution generally turns yellow after 1 h, indicating formation of silver nanoparticles. UV-vis absorption spectrum of the sample after 20 h shows surface plasmon absorption of these Ag(0) particles with max wavelength at 419 m. Expected particle size distribution would be 5.3+/−2.6 nm.

In some preferred embodiments, the suspensions of dispersed silver nanoparticles in starch are highly stable and show no signs of aggregation after 2 months of storage. The use of environmentally benign materials for reducing and protecting agents offers ready integration to biologically relevant systems.

In some preferred embodiments, aqueous solutions of silver nitrate salt, sodium bicarbonate and tannic acid are mixed with adjusting the pH to 7.4, and then refluxed with stirring for an hour to complete an oxidation-reduction reaction. The final suspension is a clear dark brown suspension that is evidence of AgNPs. Expected particle size distribution would be predominately 2-5 nm.

In some preferred embodiments, the peak surface plasmon absorption of a suspension of the silver nanoparticles is from 400 to 420 nm, and most preferably about 411 nm. In some preferred embodiments, the particles have an average diameter of from 1 to 10 nanometers (NM), most preferably an average diameter of from 2 to 10 nm, and even more preferably an average diameter of from 3 to 5 nm.

Silver nanoparticles of the present invention may be delivered in any suitable format. In some embodiments, the present invention provides methods of treating, alleviating, ameliorating, or inhibiting infection by a SARS-CoV-2, or reducing symptoms or outbreaks associated with infection by SARS-CoV-2, or treating COVID-19 in a subject in need thereof comprising administering an effective concentration of silver nanoparticles. In some preferred embodiments, an effective concentration of silver nanoparticles is from about 1 to 200 μg/ml, more preferably from 1 to 100 μg/ml, and most preferably from 5 to 50 μg/ml in suspension such as an aqueous suspension or other formulation as described herein, for example, for treating, alleviating, ameliorating, reducing or inhibiting infection by SARS-CoV-2 or symptoms associated with SARS-CoV-2. In other preferred embodiments, the concentration of silver nanoparticles in the suspension is from 0.01 to 200 μg/ml. In some preferred embodiments, the concentration of silver nanoparticles in the suspension is from 0.01 to 100 μg/ml. In some preferred embodiments, the concentration of silver nanoparticles in the suspension is from 0.01 to 10 μg/ml. In some preferred embodiments, the concentration of silver nanoparticles in the suspension is from 0.01 to 5 μg/ml. In some preferred embodiments, the concentration of silver nanoparticles in the suspension is from 0.1 to 200 μg/ml. In some preferred embodiments, the concentration of silver nanoparticles in the suspension is from 0.1 to 100 μg/ml. In some preferred embodiments, the concentration of silver nanoparticles in the suspension is from 0.1 to 10 μg/ml. In some preferred embodiments, the concentration of silver nanoparticles in the suspension is from 0.1 to 5 μg/ml.

In some preferred embodiments, the dosage for inhalation is from 0.5 ml to 10 ml of a aqueous suspension of from 10 to 200 μg/ml silver nanoparticles. In some preferred embodiments, the dosage for inhalation is from 1.0 ml to 5.0 ml of a aqueous suspension of from 50 to 200 μg/ml silver nanoparticles. In some preferred embodiments, the dosage for inhalation is from 2.0 ml to 4.0 ml of a aqueous suspension of from 75 to 100 μg/ml silver nanoparticles. In some preferred embodiments, the dosage for inhalation is about 3.0 of a aqueous suspension of about 100 μg/ml silver nanoparticles. In some preferred embodiments, the dosage is administered from 1 to 5 times daily. In some preferred embodiments, the dosage is administered 3 times daily. In some preferred embodiments, the dosage is administered for from 5 to 20 days. In some preferred embodiments, the dosage is administered for from about 7 to 14 days.

In some preferred embodiments, the administration of the silver nanoparticle composition is sufficient to relieve or ameliorate one or more symptoms of COVID-19 in a subject. The symptoms that are relieved or ameliorated include one or more of fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory. In some preferred embodiments, administration of the silver nanoparticle composition improves or alleviates one or more of the following symptoms in a long COVID patient: fatigue, loss of smell and taste, shortness of breath, cough, joint pain, chest pain, difficulty with thinking and concentration (sometimes referred to as “brain fog”), depression, headache, heart palpitations, inflammation of the heart muscle, rash, hair loss, sleep disorders, loss of lung function and loss of memory.

In some embodiments, the silver nanoparticles are provided in an aqueous suspension, including gels, suitable for use as a spray or mist. In some embodiments, the aqueous silver nanoparticles suspension is incorporated into a pump-spray container, such as precompression pump, or a device such as a nebulizer or cold mist system, for delivery into the nose, mouth or lungs as a fine mist or spray.

In some preferred embodiments, the present invention provides a spray bottle configured for application of a nasal spray to the nose of animal or human containing any of the compositions described above. In some embodiments, the silver nanoparticle formulation of the present invention contain a pharmaceutically acceptable excipient which is effective in forming a thixotropic suspension of the solid particles of medicament comprising the silver nanoparticles, such as those described in U.S. Pat. No. 7,122,206. The excipient is preferably present in an amount which maintains the particles of medicament suspended in the composition during non-use and during spray of the composition into the nasal cavity, and also when the composition is deposited on the mucosal surfaces of the nasal cavities or endothelial surfaces in the nasal cavity or elsewhere in the body. In some embodiments, the viscosity of the composition at rest is relatively high, for example, about 400 to about 1000 cp. As the composition is subjected to shear forces, for example, upon being subjected to forces involved in its being agitated before spraying, the viscosity of the composition decreases (for example, to about 50 to about 200 cp) and it flows readily through the spray device and exits therefrom in the form of a fine plume which infiltrates and deposits on the mucosal surfaces of at least the following parts of the nose: the anterior regions of the nose (frontal nasal cavities); the frontal sinus; the maxillary sinuses; and the turbinates which overlie the conchas of the nasal cavities. Thus, in some preferred embodiments, the silver nanoparticle formulations of the present invention comprise a freely flowable liquid, and in sprayed form, a fine mist that finds its way to and deposits on the desired mucosa. In deposited and relatively unstressed form, the composition increases in viscosity and assumes its gel-like form which includes particles of the medicament suspended therein and which resists being cleared from the nasal passages by the inherent mucocillary forces that are present in the nasal cavities.

Any pharmaceutically acceptable material which is capable of maintaining the solid particles of medicament dispersed substantially uniformly in the composition and of imparting to the composition desired thixotropic properties can be used. Such material is referred to as a “suspending agent”. Examples of suspending agents include carboxmethylcellulose, veegum, tragacanth, bentonite, methylcellulose, and polyethylene glycols. A preferred suspending agent is a mixture of microcrystalline cellulose and carboxymethylcellulose, the former being present preferably in a major amount, most preferably in an amount of about 85 to about 95 wt. %, with the latter constituent comprising about 5 to about 15 wt. % of the mixture.

The amount of suspending agent comprising the composition will vary depending on the particular medicament and amount used, the particular suspending agent used, the nature and amounts of the other ingredients comprising the composition, and the particular viscosity values that are desired. Generally speaking, it is believed that the most widely used compositions will comprise about 1 to about 5 wt. % of the suspending agent.

The silver nanoparticle formulations of the present invention can preferably include other ingredients which impart desired properties to the composition. In some embodiments, dispersing or wetting agents are utilized. Any dispersing agent which is effective in wetting the particles and which is pharmaceutically acceptable can be used. Examples of dispersing agents that can be used are fatty alcohols, esters, and ethers, including, for example, those sold under the trademarks Pluronic, Tergitol, Span, and Tween. It is preferred to use a hydrophilic, non-ionic surfactant. Excellent results have been achieved utilizing sorbitan monooleatepolyoxyethylene which is available under the trademark Polysorbate 80.

In some embodiments, the compositions comprise an anti-oxidant. Examples of pharmaceutically acceptable anti-oxidants that can be used in the composition include ascorbic acid, sodium ascorbate, sodium bisulfite, sodium thiosulfate, 8-hydroxy quinoline, and N-acetyl cysteine. It is recommended that the composition comprise about 0.001 to about 0.01 wt. % of the anti-oxidant.

Also, for stability purposes, the silver nanoparticle formulations should be protected from microbial contamination and growth. Examples of pharmaceutically acceptable anti-microbial agents that can be used in the composition include quaternary ammonium compounds, for example, benzalkonium chloride, benzethonium chloride, cetrimide, and cetylpyridinium chloride; mercurial agents, for example, phenylmercuric nitrate, phenylmercuric acetate, and thimerosal; alcoholic agents, for example, chlorobutanol, phenylethyl alcohol, and benzyl alcohol; antibacterial esters, for example, esters of para-hydroxybenzoic acid; and other anti-microbial agents such as chlorhexidine, chlorocresol, and polymyxin. It is recommended that the composition comprise about 0.001 to about 1 wt. % of the anti-microbial agent.

The formulations of the present invention includes preferably an iso-osmotic agent which functions to prevent irritation of nasal mucosa by the composition. Dextrose in anhydrous form is a preferred iso-osmotic agent. Examples of other pharmaceutically acceptable iso-osmotic agents which can be used include sodium chloride, dextrose and calcium chloride. It is recommended that the composition comprise up to about 5 wt. % of the iso-osmotic agent.

The silver nanoparticle formulations of the present invention can be prepared in any suitable way. In preferred form, an aqueous suspension of the solid particles of medicament and dispersing agent is formed and combined with an aqueous suspension which contains the suspending agent. The former is preferably prepared by adding the medicament to an aqueous solution of the dispersing agent and mixing thoroughly. The latter is prepared by acidifying the water (pH about 4.7 to about 5.3) prior to adding the suspending agent. In particularly preferred form, an aqueous solution of the quaternary compound (anti-microbial agent) is added to the aqueous suspension of medicament, and the other ingredients (for example, iso-osmotic agent, anti-oxidant or chelating agent) are added to the thixotropic suspension. Each of the aforementioned batches of composition is mixed thoroughly before being combined. The preferred means of combining the batches of composition is to introduce one of the batches, preferably the “medicament” batch into the bottom of the other batch, for example, by pumping the batch upwardly through the other batch. The composition comprising the combined batches is mixed thoroughly. Use of the preferred method of preparation provides an efficient and effective way for formulating a composition that has the solid particles of medicament substantially uniformly dispersed therein while avoiding problems that are generally associated with the preparation of water-based pharmaceutical compositions, for example, excessive foaming and non-uniformity of the particle dispersement.

The amount silver nanoparticle formulation applied to each of the nasal passages will vary depending on the nature of the condition being treated and the nature of the individual being treated. In some preferred embodiments, the daily dosage of silver nanoparticle formulation is delivered in from 1 to 8 administrations per day. Accordingly, the present invention provides an article of manufacture comprising a spray bottle having a silver nanoparticle formulation therein for delivery into a body cavity such as the nose. The spray bottle may preferably comprise a pump system for expelling the silver nanoparticle formulations from the bottle, such as a compression mump, spray pump or precompression pump.

In some embodiments, the silver nanoparticles are provided in a fluid that can be used for atmospheric treatment, such as by a mist. In some embodiments, the present invention provides a device comprising a reservoir, a pump, and a nozzle, wherein the reservoir comprises a fluid (e.g., a suspension) comprising silver nanoparticles that can be expelled via the pump through the nozzle to provide a mist comprising the silver nanoparticles. In some embodiments, the device is a nebulizer, while in other embodiments, the device is an automated mist dispenser.

In some embodiments, the silver nanoparticles are provided as an aerosol spray in an appropriate aerosol spray dispensing device. Accordingly, in some embodiments, the present invention provides a device or composition comprising silver nanoparticles and an aerosol propellant. Propellants include, but are not limited to, mixtures of volatile hydrocarbons, typically propane, n-butane and isobutene, dimethyl ether (DME), methyl ethyl ether, nitrous oxide, carbon dioxide and hydrofluoroalkanes (HFA): either HFA 134a (1,1,1,2,-tetrafluoroethane) or HFA 227 (1,1,1,2,3,3,3-heptafluoropropane) or combinations of the two. Typically, the silver nanoparticle suspension will be miscible with the propellant.

In some preferred embodiments, aerosols of the invention are made by nebulizing the nanoparticle containing suspension using a variety of known nebulizing techniques. Perhaps the simplest of systems is the “wo-phase” system which consists of a solution or a suspension of active ingredient, in the present case, silver nanoparticles, in a liquid propellent. Both liquid and vapor phases are present in a pressurized container and when a valve on the container is opened, liquid propellent containing the nanoparticle dispersion is released. Depending on the nature of the ingredients and the nature of the valve mechanism, a fine aerosol mist or aerosol wet spray is produced.

There are a variety of nebulizers that are available to produce the aerosols of the invention including small volume nebulizers. Compressor driven nebulizers incorporate jet technology and use compressed air to generate the aerosol. Commercially available devices are available from Healthdyne Technologies Inc; Invacare Inc.; Mountain Medical Equipment Inc.; Pari Respiratory Inc.; Mada Mediacal Inc.; Puritan-Bennet; Schuco Inc.; Omron Healthcare Inc.; DeVilbiss Health Care Inc; and Hospitak Inc. Ultrasonic nebulizers deliver high medication output and are used by patients-suffering from severe asthma, or other severe respiratory related illnesses. These various types of nebulizers may be used as delivery devices for silver nanoparticles.

In other embodiments, the silver nanoparticles are provided in a nasal spray. In some preferred embodiments, the nasal sprays comprise one or more excipients selected from a buffer, a solubilizer, a preservative, an antioxidant, a humectant, a surfactant, a bioadhesive polymer, and a penetration enhancer. Examples of buffers useful in nasal sprays of the invention include sodium phosphate, sodium citrate, and citric acid. Examples of solubilizers useful in nasal sprays of the invention include solvents or co-solvents such as glycols, alcohol, Transcutol (diethylene glycol monoethyl ether), medium chain glycerides, Labrasol (saturated polyglycolyzed C8-C10 glyceride) and cyclodextrin. Examples of preservatives useful in nasal sprays of the invention include parabens, phenyl ethyl alcohol, benzalkonium chloride, EDTA and benzoyl alcohol. Examples of antioxidants useful in nasal sprays of the invention include sodium bisulfite, butylated hydroxytoluene, sodium metabisulfite and tocopherol. Examples of humectants useful in nasal sprays of the invention include glycerin, sorbitol and mannitol. Examples of surfactants useful in nasal sprays of the invention include polysorbet as well as surfactants described elsewhere herein.

In some preferred embodiments, the pH of a nasal spray comprising PBP of the present invention is from pH 5.0 to 6.5. In some preferred embodiments, the osmolality of a nasal spray comprising PBP of the present invention is from 100 or 600 mOsmol/Kg

In some embodiments, the nasal spray comprising a PBP composition is provided in a metered-dose spray pump. Metered spray pumps of the present invention pumps preferably deliver 100 μl (25-200 μl) per spray. In some preferred embodiments, the device comprises a nozzle insertable into the nasal cavity.

### EXAMPLES

### Example 1

To prepare silver nanoparticles, a 0.1M solution of silver nitrate and a 0.17 wt % aqueous solution of soluble starch are prepared. A 100 uL aliquot of silver nitrate solution is added to 6 mL starch solution. After complete dissolution, a 150 uL aliquot of 0.1M aqueous solution of β-D-glucose is added with stirring. The mixture is heated to 40° C. and maintained at this temperature for 20 h. All solution components are preferably purged with argon before use and reduction proceeds in presence of argon to eliminate oxygen. The suspension turns yellow after 1 h, indicating formation of silver nanoparticles. UV-vis absorption spectrum of the sample after 20 h is expected to show a surface plasmon absorption of these Ag(0) particles with max wavelength at 419 m. Expected particle size distribution is 5.3+/−2.6 nm.

### Example 2

In vitro study: Investigating the antiviral property of AgNPs in inhibiting viral infection in vitro. A549 cells, a human alveolar type II-like epithelial cell line, and HEp-2 Cells will be cultured in F12K and MEM, respectively, supplemented with 10% (vol/vol) FBS, 10 mM glutamine, 100 IU/mL penicillin, and 100 μg/mL streptomycin. Confluent monolayers will be infected with virus incubated with varying doses of AgNPs (0, 10, 25 or 50 μg/mL) with shaking for 1 h at room temperature prior to plating. A549 cells and HEp-2 cells will be infected at a multiplicity of infection (MOI) of 1 for 24 h and 0.01 for 48 h, respectively. After infection, supernatants will be aliquoted and stored at −80 C. To evaluate viral titer, serial five-fold dilutions of infected supernatants will be determined by plaque assay under methylcellulose overlay. Plaques will be visualized five days later, and viral titers will be calculated as PFU/mL. Lactate dehydrogenase (LDH) cytotoxicity assay will be performed on supernatants to measure the cellular damage.

### Example 3

In vivo study: Investigating the antiviral property of AgNPs in inhibiting viral infection in vivo. Female, 10 to 12-week-old BALB/c mice will be purchased from Jackson Laboratory and housed under pathogen-free conditions in the animal research facility. A mixture of Ketamine (90-150 mg/kg) and Xylazine (7.5-16 mg/kg) will be administered by intraperitoneal (IP) injection for anesthesia and euthanasia. The dosage of AgNPs will be calculated based on the weight of the animals. All inoculants will be incubated with shaking for 1 h at room temperature prior to inoculation. Under light anesthesia, mice will be intranasally inoculated with 100 μL of sterile PBS as a mock inoculation (Negative Control 1), AgNPs (2 mg/kg or 4 mg/kg) diluted in PBS (Negative Control 2), virus diluted in PBS at a dose of 5×106 PFU (Positive Control), virus mixed with AgNPs (2 mg/kg or 4 mg/kg) diluted in PBS (Treatment). Animals from all groups will be evaluated on a daily basis for weight loss, illness score, and presence of respiratory symptoms. The percentage of bodyweight change will be plotted over time. Clinical illness scores will be visually determined by two investigators using a standardized 0-5 grading system (0-no disease, 1-slightly ruffled fur, 2-full ruffled fur, 3-ruffled fur and hunched back, 4-ruffled fur, hunched back and inactive, and 5-death). These parameters have been shown to closely correlate with lung pathology in experimental infection of mice.

Cytokines, chemokines, interferons, and elastase will be all measured using Bronchoalveolar Lavage Fluid (BALF) collected at day one and day 5 post infection (p.i.) Total proteins will be measured using BALF collected at days one and five p.i. Levels of cytokines and chemokines in the BALF will be determined with a Bio-Plex Pro Mouse Group I 23-plex panel (BioRad Laboratories, Hercules, Calif., USA). Interferon (IFN)-α and IFN-β will be measured by ELISA, following the manufacturer's protocol (PBL Biomedical Laboratories, Piscataway, N.J., USA). Total protein concentrations will be determined using the Bradford method (BioRad Laboratories, Hercules, Calif., USA). Neutrophil elastase will be measured using a neutrophil elastase ELISA kit (R&D Systems, Minneapolis, Minn., USA). Absorbance for all microplate assays will be measured on a microplate reader.

### Example 4

Mouse study: Mortality with virus and test the efficacy of silver nanoparticles in inhibiting viral infection and replication in lungs. Thirty-six, 8 to 10-week-old, female BALB/c mice will be purchased from Jackson Laboratory and housed under pathogen-free conditions in the animal research facility. A mixture of Ketamine (90-150 mg/kg) and Xylazine (7.5-16 mg/kg) will be administered by intraperitoneal (IP) injection for anesthesia. Under light anesthesia, mice will be intranasally inoculated with 20 μL of the viral titers. Mice will be divided into four groups (N=9/each group)—(1) virus only (positive control), (2) sterile PBS as a mock inoculation (Negative Control 1), (3) treated with AgNPs diluted in PBS (Treatment), and (4) treated with Oseltamivir (a neuraminidase inhibitor based antiviral agent widely used against influenza). After 24 h infection with virus, AgNPs and Oseltamivir will be administered to the anesthetized mice via intranasal absorption at concentrations of 5 mg/kg and 20 mg/kg mice body weight, respectively. Antiviral treatments will be repeated daily for next 2 days. Clinical signs, changes in body weight, and mortality will be recorded daily up to day 14.

Following sacrifice of the mice, three mice will be chosen at random and their lungs will be weighed for calculating lung index using the equation, (weight of the lung/weight of the mouse)×100%. Lung homogenates will be centrifuged at 10,000 g for 10 minutes before the supernatant will be collected for determination of virus titer by the standard hemagglutinin assay.

For lung histology, the middle lobe of the right lung from each mouse will be removed and fixed in 10% formaldehyde solution for 24-48 hours. The tissues will be dehydrated in a graded ethanol series and embedded in paraffin. Sections will be embedded in wax and cut into 5 μm slices for hematoxylin and eosin (H&E) staining, and pathologic changes will be studied by light microscopy. In particular, histopathology slides will be observed for changes in the structure of pulmonary alveolus, lymphocytic infiltration, and alveolar wall necrosis.

This experiment will be repeated using viral titers recovered from lung homogenates recovered from mice in the first experiment. Clinical signs, changes in body weight, and mortality will be recorded daily up to day 14. Briefly, at Day 6 in the first experiment, three mice from each group will be euthanized by isoflurane overdose. The lungs will be extracted, washed in 2 mM ethylenediaminetetraacetic acid (EDTA) phosphate-buffered saline, and kept at −80° C. until further experiments. Parts of lung tissue will be homogenized and administered intranasally for the second passage of mouse infection.

### Example 5

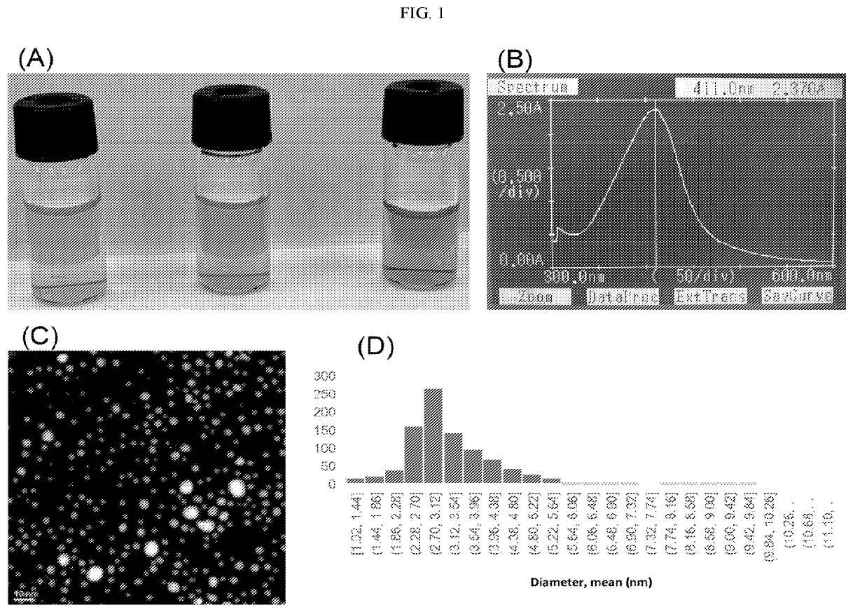

Colloidal solutions of stabilized AgNPs (silver nanoparticles) may also be prepared as follows. Aqueous solutions of silver nitrate salt (2 mL, 100 mM), sodium bicarbonate (0.4 mL, 120 mM) and tannic acid (2 mL, 5.8 mM) are mixed with adjusting the pH to 7.4, and then refluxed with stirring for an hour to complete an oxidation-reduction reaction. The final suspension is a clear dark brown suspension that is evidence of AgNPs. (See FIG. 1a).

The suspension is tested through several CMC controls (See FIG. 1). The UV-vis absorption spectrum of the suspension has a narrow single surface plasmon peak at ˜411 nm wavelength, suggesting a very narrow size distribution of spherical AgNPs in the suspension. (See FIG. 1b).

Further characterization is done using dynamic light scattering and transmission electron microscopy (TEM). The AgNPs are confirmed in TEM images to be spherical and predominantly 3-5 nm in size (See FIG. 1c), with a narrow size distribution (See FIG. 1d). The AgNPs suspension is stored in dark at room temperature and has been documented to be stable over 6 months at room temperature without particle precipitation.

### Example 6

This example provides data related to the antiviral and cytotoxic properties of silver nanoparticles against SARS-CoV2 using an immunofluorescence-based assay. The silver nanoparticles used in this Example were made according to Example 5.

To determine the antiviral activity of silver nanoparticles two procedures were followed. Silver nanoparticles were either mixed with SARS-CoV2 for 2 h prior to addition to the cells or added to Vero cells for 2 h after infection. Infection was performed for 1 h, virus removed by washing, and cells incubated for 24 h prior to analysis. Silver nanoparticles were tested at multiple dilutions ranging from 40 μg/ml to 0 μg/ml. Antiviral activity was determined at 24 h using an immunofluorescence-based assay. Cytotoxicity was determined using an MTT assay on uninfected cells treated with the same concentrations of nanoparticles. Remdesivir was included as an assay control.

Using an immunofluorescence-based assay at 24 h after infection, silver nanoparticles showed antiviral activity against SARS-CoV2 with an EC50 between 0.1 μg/ml (when administered pre infection) and 2.3 μg/ml (when administered post infection), and a selectivity index of 54.8 or 4.5, respectively. The corresponding EC90 and SI90 values were 3.4 μg/ml and 2.5 μg/ml (EC90) and 17.1 and 16.9 (SI90). Cytotoxicity was observed at concentrations above 5 μg/ml.

Silver nanoparticles display antiviral activity against SARS-CoV2, with EC50 and EC90 values in the low microgram/ml range. Cytotoxicity was observed at concentrations above 5 μg/ml.

### Experimental Procedure

The antiviral activity of 7 dilutions of silver nanoparticles was explored following two modes of administration: incubation with the virus before infection (pre-infection), and incubation with the cells after infection (post-infection) with SARS-CoV2. Cells were infected with virus for 1 h, washed, and the cells cultured for 24 h. The cytotoxicity of the same range of concentrations of silver nanoparticles was determined by MTT assay.

Cell Plating

Cells were cultured in Complete media: M199 medium supplemented with 5% FBS. Cells were detached and counted following SOP-RA 003 and SOP-RA 004. Count was recorded in the Cell Count Logbook, Volume 1, 27/01/2021. Cells were seeded in complete media at 8,000 cells/1000/well in two plates: one for the cytotoxicity assay and one for the infectivity assay. After seeding, the plates were incubated at RT for 5 minutes for even distribution, and then at 37° C., 5% CO₂ until the following day.

Virus Dilutions

The virus stock was diluted to bring the concentration to 1×10⁶ TCID50/ml. 2 μl of diluted virus was transferred into 5000 μl of supplemented 0.4% BSA media (MOI 0.002, for Remdesivir). 20 μl of diluted virus was transferred into 5000 μl of supplemented 0.1% BSA media (MOI 0.02 for nanoparticles). Media was removed from the cells and 50 μl virus (MOI 0.02) was used for columns 1-6. Media was removed from the cells and 50 μl virus (MOI 0.002) was used for columns 7-10 (Remdesivir 7-9 and Infected control 10). Column 11 (uninfected control).

Silver Nanoparticle Dilutions

The initial stock 1600 μg/ml was diluted with water as follows:
a 60 μl of silver nanoparticles were added to 0 μl of H₂O and 1140 μl supplemented media (0.1% BSA) to achieve 80 μg/ml concentration;
a 30 μl of silver nanoparticles were added to 30 μl of H₂O and 1140 μl supplemented media (0.1% BSA) to achieve 40 μg/ml concentration;
a 15 μl of silver nanoparticles were added to 45 μl of H₂O and 1140 μl supplemented media (0.1% BSA) to achieve 20 μg/ml concentration;
a 7.5 μl of silver nanoparticles were added to 52.5 μl of H₂O and 1140 μl supplemented media (0.1% BSA) to achieve 10 μg/ml concentration;
a 3.76 μl of silver nanoparticles were added to 56.24 μl of H₂O and 1140 μl supplemented media (0.1% BSA) to achieve 5 μg/ml concentration;
a 1.88 μl of silver nanoparticles were added to 58.12 μl of H₂O and 1140 μl supplemented media (0.1% BSA) to achieve 2.5 μg/ml concentration;
a 0 μl of silver nanoparticles were added to 60 μl of H₂O and 1140 μl supplemented media (0.1% BSA) to achieve 0 μg/ml concentration.

Remdesivir Dilutions

The initial stock 10 mM was diluted as follows: a 3 μl of the initial stock was diluted with 747 μl supplemented media (0.4% BSA) to achieve 40 μM working solution.

Cell Treatment

### Pre-Infection

Each concentration of silver nanoparticles was mixed 1:1 with virus (infectivity assay), or media supplemented with 0.1% BSA (cytotoxicity assay) in triplicate for 2 h at 37° C. After incubation, cells were washed with supplemented media (0.1% BSA) and in column 1, 2, and 3, 100 μl of virus (MOI 0.02) (immunofluorescence) or media (cytotoxicity)+silver nanoparticle pre-incubated samples were transferred to the cells for 1 h at 37° C. At the end of the incubation, cells were washed with supplemented media (0.1% BSA) and cultured for 24 h at 37° C.

### Post Infection

Supplemented media (0.1% BSA) was mixed 1:1 with virus (MOI 0.02), and 100 μl added to cells in columns 4, 5, and 6, for 1 h at 37° C. After infection, each concentration of silver nanoparticles was mixed 1:1 with supplemented media (0.1% BSA) in triplicate. Cells were washed with supplemented media (0.1% BSA) to remove virus, and 100 μl of silver nanoparticles were added to the cells for 2 h at 37° C. At the end of the incubation, cells were washed with supplemented media (0.1% BSA) and cultured for 24 h from infection at 37° C.

### Controls

Remdesivir (40 μM working solution) was serially diluted 3-fold for an 8 step dilution series. Diluted Remdesivir was mixed 1:1 with virus (infectivity assay) or supplemented media (0.4% BSA, cytotoxicity assay) in triplicate. Cells were washed with supplemented media (0.4% BSA) and in column 7, 8, and 9, 100 μl of Remdesivir+virus (MOI 0.002, infectivity assay) or supplemented media (cytotoxicity assay) were transferred to the cells for 24 h at 37° C. An untreated infected and untreated and uninfected control were included in row 10 and 11, respectively.

Fixation and Development

After 24 h, one plate was washed with PBS, fixed for 30 mins with 4% formaldehyde, washed again with PBS, and stored in PBS at 4° C. until staining. The cytotoxicity plate was treated with MTT to determine cell viability.

Infectivity Readout

Cells were immunostained following SOP-RA 005. Briefly, any residual formaldehyde was quenched with 50 mM ammonium chloride, after which cells were permeabilised (0.1% Triton X100) and stained with an antibody recognizing SARS-CoV2 spike protein (GeneTex GTX632604). The primary antibody was detected with an Alexa-488 conjugate secondary antibody (Life Technologies, A11001), and nuclei were stained with Hoechst. Images were acquired on an CellInsight high content confocal microscope (ThermoFisher) using a 10× objective, and percentage infection calculated using HCS studio (infected cells/total cells×100).

Cytotoxicity Readout

Cytotoxicity was detected by MTT assay following SOP-RA 006. Briefly, the MTT reagent (Sigma, M5655) was added to the cells for 2 h at 37° C., 5% CO₂, after which the media was removed and the precipitate solubilized with a mixture of 1:1 Isopropanol:DMSO for 20 minutes. The supernatant was transferred to a clean plate and signal read at 570 nm.

Determination of EC50 Concentration—IF Assay

Normalised percentages of inhibition were calculated using the following formula:

$$Normalised \% inhibition = 100 \times ( 1 - \frac{\% Infection Sample - \% Infection Uninfected Control}{\% Infection Infected Control - \% Infection Uninfected Control} )$$

EC50 values were extrapolated from the curves representing the best fit (non-linear regression analysis, variable slope) of the logarithm of compound concentration vs. the normalised percentages of inhibition, using GraphPad Prism (version 9).

Determination of TC50 Concentration

Percentages of cytotoxicity were calculated using the following formula:

$$\% cytotoxicity = 100 - ( 100 \times \frac{Absorbance Sample}{Absorbance Untreated Control} )$$

TC50 values were extrapolated from the curves representing the best fit (non-linear regression analysis, variable slope) of the logarithm of compound concentration vs. the normalised percentages of cytotoxicity, using GraphPad Prism (version 9).

### Results

Table 1 displays the EC50, EC90, TC50, TC90 and Selectivity Index (SI) 50 and SI90 for silver nanoparticles (MOI 0.02) and the Remdesivir control (MOI 0.002).

Table 2 shows the percentage of Vero cells infected by SARS-CoV2 after incubation with silver nanoparticles or Remdesivir (assay control) for 24 h. Seven dilutions were tested as indicated in the table. Three technical replicates were performed. Untreated infected and untreated uninfected controls were included.

Inhibition of SARS-CoV2 infection was observed in cells treated with silver nanoparticles, with EC50 of 0.1 μg/ml (preinfection) and 2.3 μg/ml (post-infection). EC90 for the same experimental conditions was 3.4 μg/ml and 2.5 μg/ml, respectively. For the pre-infection mode, the EC50 values is extrapolated, as the concentrations tested did not allow to observe a progressive decrease of inhibitory effect over additional dilutions.

High cytotoxicity was observed at concentrations above 5 μg/ml, with a TC50 value of 6.1 μg/ml (pre-infection) and 10.3 μg/ml (post-infection). TC90 for the same experimental conditions was 58.3 μg/ml and 42.2 μg/ml, respectively. The SI50 was 54.8 (pre-infection) and 4.5 (post-infection), and the SI90 was 17.1 (pre-infection) and 16.9 (post-infection).

TABLE 1

| Test article | EC50 (μg/ml) | TC50 (μg/ml) | SI50 | EC90 (μg/ml)) | TC90 (μg/ml) | S190 |
|---|---|---|---|---|---|---|
| Stiver nanopartices- | 0.1124 | 6.105 | 54.80 | 3412 | SS 372 | 17.108 |
| pre infection | | | | | | |
| Silver nanopartices- | 2.284 | 10.27 | 1.4% | 2.495 | 42.153 | 16.895 |
| post-infection | | | | | | |
| Test artide | EC50 (μM) | TC50 (μM) | SI50 | EC90 (μM) | TC90 (μM) | S190 |
| Revndesivir- with | 0. 6539 | 277541304 | 420580852 | 1687 | 3.4416 | 204*16 |
| infection | | | | | | |

EC50, TC50, EC90, TC90, and S150 (=TC50/EC50) and S190 (TC90/EC90) values for each tested compound.

TABLE 2
Percentages of infection at 24 h.

| Test | | | | | | |
|---|---|---|---|---|---|---|
| Compound | | | | | | |
| (μg/ml) | Pre-infection_0.02 | | | Post-Infection_0.02 | | |
| 40 | 0.00 | 0.00 | 0.38 | 2.54 | 2.05 | 1.76 |
| 20 | 0.81 | 0.70 | 1.58 | 5.26 | 1.90 | 2.02 |
| 10 | 0.78 | 0.48 | 1.12 | 0.29 | 0.42 | 1.19 |
| 5 | 3.40 | 2.90 | 1.39 | 0.27 | 0.29 | 0.32 |
| 2.5 | 1.68 | 1.43 | 0.72 | 2.44 | 1.92 | 1.91 |
| 1.25 | 3.52 | 2.25 | 4.28 | 28.82 | 20.44 | 15.81 |
| 0 | 14.60 | 14.02 | 22.02 | 31.43 | 17.58 | 11.31 |
| Remdesivir | | | | | | |

| (μM) | Remdesivir_0.002 | | | Infected | Uninfected |
|---|---|---|---|---|---|
| 20.00 | 0.78 | 0.24 | 0.27 | 28.65 | 0.09 |
| 6.67 | 0.34 | 0.27 | 0.23 | 12.56 | 0.39 |
| 2.22 | 2.04 | 1.54 | 1.69 | 13.81 | 0.11 |
| 0.74 | 10.55 | 6.40 | 14.40 | 31.82 | 0.42 |
| 0.25 | 8.05 | 17.17 | 39.50 | 18.84 | 0.19 |
| 0.08 | 37.97 | 32.40 | 10.00 | 10.12 | 0.29 |
| 0.03 | 20.60 | 10.69 | 23.44 | 28.05 | 0.11 |
| 0.01 | 17.52 | 14.77 | 19.44 | 41.80 | 0.10 |

### CONCLUSIONS

Under the conditions tested, silver nanoparticles displayed antiviral activity against SARS-CoV2 at 24 h post infection, with EC50 values of 0.1-2.3 μg/ml (SI50 of 54.8-4.5) and EC90 values of 14-2.5 μg/ml (SI90 of 17.1-16.9). Significant cytotoxicity was observed at concentrations above 5 μg/ml. Silver nanoparticles were more potent when added to the virus pre-infection, however, this was also accompanied by a higher cytotoxicity. When silver nanoparticles are added post-infection, 90% inhibition of infectivity is reached at concentrations of only marginally higher that the EC50 value.

### Example 7

The treatment methods of the present invention contemplate aerosolization of an aqueous suspension of stabilized AgNPs via a continuous nebulizer into lungs for the treatment of infections by respiratory viruses including but not limited to SARS CoV 2 (i.e., treatment of COVID-19 disease). It is contemplated that such inhaled AgNPs would attach to respiratory viruses lodged in the upper and lower respiratory tracts, disrupt the viral morphological structure, block spike proteins from binding to receptors of host cells, and inhibit viral infection and replication.

Nebulized drug delivery offers numerous advantages compared to oral ingestion or IV injections. The aerosolized drug formulation provides an immediate contact with upper and lower respiratory tracts. It is a non-invasive technique for successful targeting of different regions of the lungs. It reduces the adverse drug reactions as compared to conventional drug therapies.

Based on a review of several published studies, the minimal inhibitory concentration (MIC) of antiviral AgNPs in vitro is about 10 μg/ml. Mucus volume in the lining of bronchial tree is about 1 mL [24]. Therefore, the target is to deliver 10 μg of AgNPs in the pulmonary region comprised of bronchial tree.

Respiratory infections increase mucus production by 3× in the pulmonary region. Consequently, as common in antibiotic clinical inhalation therapies [19], the target dose for deposition in bronchial tree should be 3× of MIC, i.e. about 30 μg AgNP.

Commonly available hand-held continuous nebulizers aerosolize 5 μm size water droplets. They are effective in targeting bronchial alveolar region of the lungs. Under oral breathing of 5 μm aerosol droplets, deposition in bronchial tree is about 30%. Therefore, the inhaled delivery dosage should be adjusted by a 3.33× factor to 100 μg AgNP to reach target dose in bronchial tree mucus lining.

It is a common practice to nebulize 3 mL suspension of drugs for inhalation delivery. It is a well-tolerated volume. Hence, to inhale 100 μg AgNP in 3 mL solution, the colloidal suspension should be 33.33 μg/mL. However, when using a continuous nebulizer, only a one third of the dosage is inhaled, as inhalation is only one third of the breathing cycle. Therefore, the concentration of aqueous suspension will be adjusted by 3× factor to 100 μg/mL.

Based on these calculations, it is preferred to nebulize 3 mL of a 100 μg/mL aqueous suspension of antiviral AgNPs. Since only one third of the dosage will be inhaled using a continuous nebulizer, it would result in inhalation of about 100 μg AgNPs in each dose. It is further preferred to nebulize 3 doses AgNPs per day, over a 10-14 day course, for antiviral therapy, as in typical antibiotics inhalation treatment plans. This amounts to daily inhalation of about 300 μg of AgNPs, for 10-14 consecutive days.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A method for treating or inhibiting infection by a respiratory virus, in a human or animal subject, the method comprising:
administering silver nanoparticles in an effective concentration to the subject under conditions such that infection by the respiratory virus is inhibited or treated.

2. A method for prophylaxis of infection by a respiratory virus, in a human or animal subject, the method comprising:
administering silver nanoparticles in an effective concentration to the subject under conditions such that infection by the respiratory virus is inhibited.

3-4. (canceled)

5. Method of claim 1, wherein the respiratory virus is selected from the group consisting of an influenza virus, respiratory syncytial virus, a parainfluenza virus, metapneumovirus, rhinovirus, a coronavirus, an adenoviruses, and a bocavirus.

6. Method of claim 5, wherein the coronavirus is SARS CoV 2 (Severe Acute Respiratory Syndrome Coronavirus 2).

7. Method of claim 1, wherein the silver nanoparticles are from 1 to 100 nm in size.

8. Method of claim 1, wherein the silver nanoparticles are provided in a formulation and the average size of the nanoparticles in the formulation are from 1 to 50 nm in size.

9. Method of claim 1, wherein the average size of the nanoparticles in the formulation is from 1 to 10 nm in size.

10. Method of claim 1, wherein the silver nanoparticles are formulated in an aqueous suspension.

11. Method of claim 1, wherein the suspension is an inhalation suspension.

12. Method of claim 11, wherein the concentration of silver nanoparticles in the suspension is from 0.01 to 200 μg/ml.

13. Method of claim 11, wherein the concentration of silver nanoparticles in the suspension is from 0.1 to 100 μg/ml.

14. Method of claim 11, wherein the concentration of silver nanoparticles in the suspension is from 0.01 to 5 μg/ml.

15. Method of claim 1, wherein the silver nanoparticles are formulated for intranasal administration.

16. Method of claim 1, wherein the silver nanoparticles are formulated with one or more physiologically acceptable carriers.

17. Method of claim 1, wherein the silver nanoparticles are stabilized to prevent agglomeration.

18. Method of claim 1, wherein the silver nanoparticles are formulated in a suspension for use in a nebulizer or misting apparatus.

19. Method of claim 1, wherein the silver nanoparticles are delivered to the lung of a subject via inhalation.

20. Method of claim 1, wherein the inhalation is via a continuous nebulizer.

21. Method of claim 1, wherein the silver nanoparticles are formulated in a suspension for use as an aerosol.

22. Method of claim 1, wherein the silver nanoparticles are formulated as a nasal spray.

23. Method of claim 22, wherein the silver nanoparticles are formulated with thixotropic agent.

24. Method of claim 21, wherein the silver nanoparticles are delivered to the lung of a subject via intranasal administration.

25. Method of claim 1, wherein a suspension of the silver nanoparticles has a surface plasmon peak between 400 and 420 nm.

26. Method of claim 1, wherein the subject is at risk for infection by SARS-CoV-2.

27. Method of claim 1, wherein the subject has COVID-19.

28. Method of claim 1, wherein the dosage for inhalation is from 0.5 ml to 10 ml of a aqueous suspension of from 10 to 200 μg/ml silver nanoparticles.

29. Method of claim 28, wherein the dosage is administered from 1 to 5 times daily.

30-31. (canceled)

32. Method of claim 1, wherein the silver nanoparticles are stabilized with starch.

33. Method of claim 1, wherein the silver nanoparticles are prepared by reduction of a silver nitrate salt with tannic acid.
